(19) United States
(12) Patent Application Publication
(10) Pub. No.: US 2025/0034063 A1
(43) Pub. Date: Jan. 30, 2025

(54) NET ZERO ETHANE CRACKER WITH NO EXTERNAL HYDROGEN IMPORT

(71) Applicants: Rian Reyneke, Katy, TX (US); John Lewis Webb, JR., Richmond, TX (US); Paul Richard Langston, Middlesex (GB)

(72) Inventors: Rian Reyneke, Katy, TX (US); John Lewis Webb, JR., Richmond, TX (US); Paul Richard Langston, Middlesex (GB)

(73) Assignee: KELLOGG BROWN & ROOT LLC, Houston, TX (US)

(21) Appl. No.: 18/786,232

(22) Filed: Jul. 26, 2024

Related U.S. Application Data
(60) Provisional application No. 63/516,104, filed on Jul. 27, 2023.

(51) Int. Cl. C07C 4/04; B01J 6/00; C10G 47/36

(52) U.S. Cl. CPC C07C 4/04; B01J 6/008; C10G 47/36

(57) ABSTRACT

A system for controlling a steam cracking furnace may include a controller configured to control an electric heater to pre-heat a combustion air feed to a radiation section of the cracking furnace, and one or more sensors configured to sense properties of a recovered pure hydrogen stream fed to the radiation section of the cracking furnace as fuel. The controller can be configured to receive the sensed properties from the one or more sensors and control the electric heater based on the one or more sensed properties.

This application claims the benefit of U.S. Provisional Application No. 63/516,104, filed Jul. 27, 2023, which is incorporated herein by reference in its entirety.

### TECHNICAL FIELD

The present disclosure relates to methods and systems for cracking ethane with low CO2 emissions using by-product hydrogen as a fuel.

### BACKGROUND

A common method of manufacturing light olefins is steam-cracking, where a hydrocarbon feed (e.g., ethane) is heated in a furnace to very high temperatures in the presence of steam. The high temperature cracks the hydrocarbons into smaller molecules, producing ethylene and a tail gas stream that includes methane (CH4), and hydrogen (H2). A portion of the tail gas stream can be used as fuel for the furnace. When the tail gas is burned in the furnace as fuel, however, a significant amount of CO2 is released because of the CH4. Combustion of H2 in the tail gas, however, has no associated CO2 emission. CH4 or other hydrocarbons should be eliminated from the tail-gas fuel as much as possible before it is burned in the furnace.

### SUMMARY

Provided herein are systems and methods to address these shortcomings of the art and provide other additional or alternative advantages.

A method of controlling a steam cracking furnace may include cracking a feed comprising hydrocarbon feedstock and steam in a furnace to create a cracked gas comprising ethylene and hydrogen, separating hydrogen from the cracked gas to create a hydrogen enriched stream, preheating a stream of combustion air, mixing the hydrogen enriched stream with the preheated combustion air stream to create a combustion mixture, and burning the combustion mixture in the furnace. The stream of air can be heated to a temperature based on a property of the hydrogen enriched stream. The combustion air stream can be heated by a heater that is external to the furnace. Heating the combustion air may include a resistive element transmitting electrical current to generate heat for heating the stream of air, receiving and processing data relating to the hydrogen enriched stream property and adjusting the electrical current in response to receiving and processing the data relating to the hydrogen enriched stream property. The property of the method may relate to a percentage of hydrogen in the hydrogen enriched stream, a flow rate of the hydrogen enriched stream, a temperature of the hydrogen enriched stream, and/or a pressure of the hydrogen enriched stream.

A system for controlling a steam cracking furnace may include a furnace comprising tubes in which a feed comprising hydrocarbon feedstock and steam is cracked to create a cracked gas comprising ethylene and hydrogen, a separator for separating hydrogen from the cracked gas to create a hydrogen enriched stream, a heater external to the furnace for heating a stream of air to a temperature based on a property of the hydrogen enriched stream. The furnace may be configured to mix and burn the hydrogen enriched stream and the preheated combustion air stream to heat the tubes. The electric heating element may include a resistive element that generates heat when the resistive element conducts electric current. The control system may include a microcontroller for processing the data based on executable instructions stored in memory. The property may relate to a flow rate of the hydrogen enriched stream, a temperature of the hydrogen enriched stream, and/or a pressure of the hydrogen enriched stream. The heater may be configured to heat the stream of air to the temperature that is based on a property of the stream of air before it is heated.

A system for controlling a steam cracking furnace may include a controller configured to control an electric heater to pre-heat a combustion air feed to a radiation section of the cracking furnace, and one or more sensors configured to sense properties of a recovered pure hydrogen stream fed to the radiation section of the cracking furnace as fuel. The controller is configured to receive the sensed properties from the one or more sensors and control the electric heater based on the one or more sensed properties.

### BRIEF DESCRIPTION OF THE DRAWINGS

The present disclosure is illustrated by way of example, and not by way of limitation, in the figures of the accompanying drawings in which like reference numerals refer to similar elements.

FIG. 1 is a schematic diagram illustrating relevant aspects of a steam-cracking furnace.

FIG. 2 is a schematic diagram illustrating relevant aspects of a hydrogen recovery system.

FIG. 3 is a schematic diagram illustrating relevant aspects of a steam-cracking furnace according to an example of the present disclosure.

### DETAILED DESCRIPTION

The following description sets forth numerous specific details such as examples of specific systems, components, methods, and so forth, to provide a good understanding of various examples of the techniques described herein.

However, it will be apparent to one skilled in the art that at least some examples may be practiced without these specific details. In other instances, well-known components, elements, or methods are not described in detail or are presented in a simple block diagram format to avoid unnecessarily obscuring the techniques described herein. Thus, the specific details set forth hereinafter are merely exemplary. Implementations may vary from these exemplary details and still be contemplated to be within the spirit and scope of the present disclosure.

Reference in the description to “an example,” “one example,” “some examples,” and “various examples” means that a particular feature, structure, step, operation, or characteristic described in connection with the example(s) is included in at least one example of the disclosure. Further, the appearances of the phrases “an example,” “one example,” “some examples,” and “various examples” in various places in the description do not necessarily all refer to the same example(s).

The description includes references to the accompanying drawings, which form a part of the detailed description. The drawings show illustrations in accordance with examples. These examples are described in enough detail to enable those skilled in the art to practice the claimed subject matter described herein. The examples may be combined, other examples may be utilized, or structural, logical, and electrical changes may be made without departing from the scope and spirit of the claimed subject matter. It should be understood that the examples described herein are not intended to limit the scope of the subject matter but rather to enable one skilled in the art to practice, make, and/or use the subject matter.

Steam-cracking furnaces burn fuel gas to provide the heat required for net process heating, to satisfy the cracking heat of reaction (endothermic reaction), and to provide waste heat for generating steam. In a typical steam-cracking furnace, about 35% of the fired duty can be used to provide the heat of reaction. About 53% of the fired duty can be used to generate superheated SHP steam (e.g., 100 barg+) and provide net feed preheat through a combination of direct heat recovery from the flue gas and indirect heat recovery through process effluent cooling in quench exchangers. About 12% of the fired duty is lost.

Fuel gas burned by furnaces varies in composition. Fuel gas used in ethane steam-cracker furnaces may contain about 80-85 mol % H2, with the remainder being mostly methane. Combustion of hydrogen has no associated CO2 emission, while combustion of methane produces approximately 230 kg CO2/Gcal of fired duty. Steam-cracking may contribute substantially to global CO2 emissions. To decarbonize the furnaces, methane or higher hydrocarbons should be eliminated or reduced as much as possible from the fuel gas.

In examples, the process and system described herein may include the use of high purity hydrogen stream as fuel gas to the furnace. In examples, the high purity hydrogen stream may be the only fuel gas for the furnace other than combustion air. In examples, the process and system described can address a duty energy balance in the system to enable the use of high purity hydrogen stream as the only fuel gas for the furnace other than combustion air. In examples, the system and method may include a combination of one or more features as described.

In examples, the method and system may include the application of a combustion air preheat. In examples, the combustion air preheat may include first and second preheating. In examples, the first air preheat may be carried out using heat from the flue gas. In examples, the second air preheat may be carried out using a heater. In examples, the heater may be an electric heater. In examples, the combustion air may be preheated to about 425° C.±15° C. in the first preheating and then up to about 725° C. in the second preheating.

In examples, the amount of preheating of the combustion air may be controlled based on one or more properties the high purity hydrogen stream used as fuel. In examples, the system and method may determine and/or anticipate the available fired duty that can be recovered from the high purity hydrogen stream. In examples, the available fired duty from the high purity hydrogen stream used as fuel may depend on properties such as flow rate, concentration, temperature, and pressure. In examples, the system and method as described may be configured to determine the amount of preheating required to the combustion air based on the combustion air properties such as flow rate, temperature, and pressure. In examples, the method and system as described may control a second preheating of the combustion air to ensure that its temperature as it is injected into the furnace is sufficient to substantially duty balance the process.

In examples, the system and method can be configured to select the combustion air preheat to balance with the available fired duty that a recovered high purity hydrogen stream can supply such that no external hydrogen import is required to carry out the steam-cracking process.

In examples, the system and method may include reducing heat recovery in Primary Quench Exchangers (PQEs) to shift more duty into a feed to effluent exchanger.

In examples, the system and method may include superheating a mixed feed stream in Secondary Quench Exchanger (SQE) against the furnace effluent leaving the primary quench exchangers.

In examples, the system and method may include a furnace burner design that may be suitable for high purity hydrogen.

In examples, the system and method may include a combination of two or more of the above features. In examples, the system and method may include a combination of all of the above features.

In examples, the method and system as described may take advantage of recovering hydrogen from the furnace effluent to use as fuel. In examples, a recovered high purity hydrogen stream may provide the only fuel source. In examples, no added fuel and/or imported hydrogen may be necessary for the steam cracking once the recovered high purity hydrogen stream is being generated. In examples, the method and system as described may provide a low or zero carbon emission system.

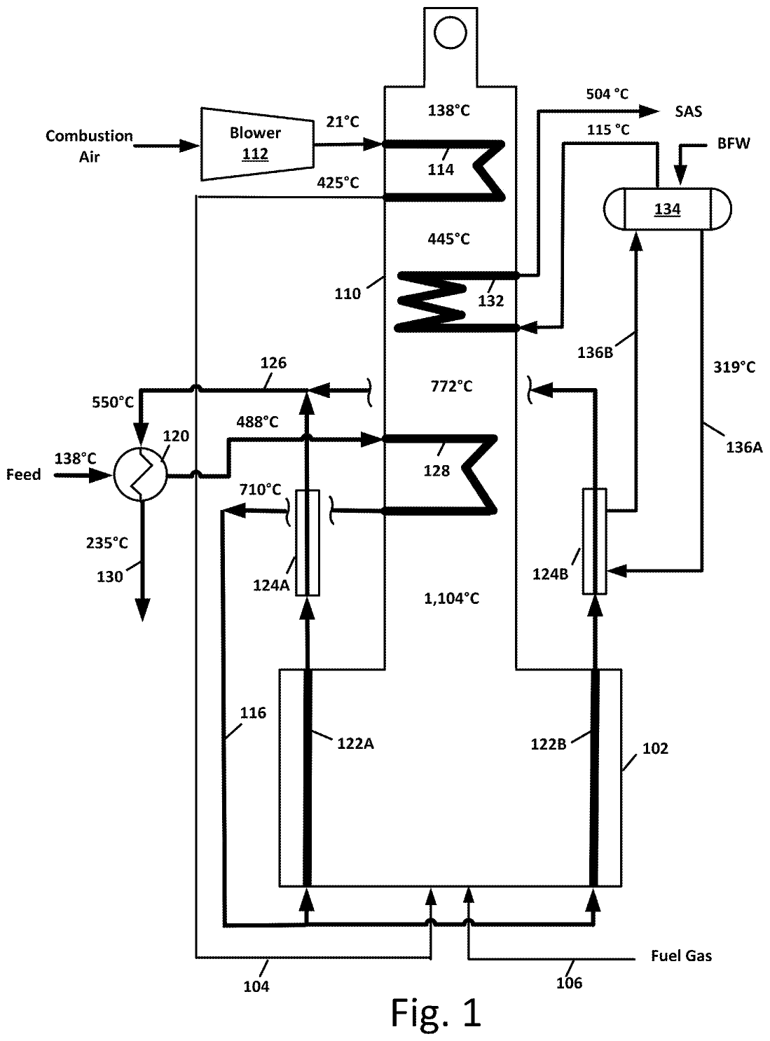

FIG. 1 is a schematic diagram illustrating some components of an example furnace system 100 that is configured for steam-cracking a hydrocarbon such as ethane. Furnace system 100 includes a radiant section (i.e., a fire box) 102 where a mixture of preheated combustion air provided by line 104 and fuel gas provided by line 106 is burnt in burners (not shown), and a convection section 110 where heat from hot flue gas can be recovered for various processes such as creating steam, as described below. Temperatures are shown at various locations within the furnace. It should be appreciated that the illustrated temperatures are only illustrative and other examples may use other temperatures.

Combustion air is provided to radiant section 102 via line 104. One way of optimizing a furnace is to preheat the combustion air. In the illustrated furnace system, combustion air is provided by a blower 112 at a temperature of 21° C. The combustion air is preheated in an air preheater (e.g., a heat exchanger) 114 to a temperature of 425° C. in the illustration using heat from the flue gas in the convection section 110. Heating the combustion air may reduce the available heat for other heating requirements that would otherwise be provided by the fired duty of the furnace burners.

A feed, comprising steam and a hydrocarbon such as ethane, enters the furnace system 100 via the feed line 116. In the illustrated example, the feed is first preheated using a feed/effluent exchanger 120, and then is further heated using a feed preheater (e.g., heat exchanger) 128 positioned within the convection section 110. According to some examples, the feed preheater 128 may heat the feed to at least 650° C. In the illustrated example, the feed is heated to 710° C. The preheated feed is then passed through the radiant section of furnace via tube banks 122A and 122B where the cracking reaction occurs. Two tubes 122 are illustrated in the drawing, but it should be appreciated that more or fewer tubes 122 may be used.

Cracked gas streams from furnace tube banks 122A and 122B are cooled in primary quench exchangers 124A and 124B. The cooled cracked gas streams are then combined in stream 126, which is used to preheat the incoming feed stream in the feed/effluent exchanger 120. Cracked gas stream 130 is processed in a recovery section as more fully described below.

In the illustrated example, the flue gas is used to heat the combustion air in the combustion air preheater 114 and the feed in the feed preheater 122. The flue gas is also used to heat steam in a steam superheater 132 to produce superheated steam (SHS). In the illustrated example, saturated steam is provided to the steam superheater 132 by heating boiler feed water (BFW) from a steam drum 134. The quench exchangers 124A and 124B can be used to heat the BFW to provide the steam, which is subsequently superheated to provide the SHS. In the illustrated example, BFW is provided to the quench exchangers 124A and 124B via line 136A and partially vaporized BFW/steam returns to the steam drum 134A via line 136B. Similar piping to and from the quench exchanger 124A is omitted for clarity.

Cracked gas 130 can be separated into ethylene, ethane, C3+ streams and tail gas that includes hydrogen and CH4. The tail gas can be used to fuel a steam-cracking furnace. Since the tail gas contains CH4, any combustion of that tail gas would generate a substantial amount of CO2. However, the tail gas can be separated to yield a stream that is enriched in H2 (and depleted in CH4). The H2-enriched stream can be used as fuel for furnace system 100 to reduce the CO2 emitted from fuel gas combustion.

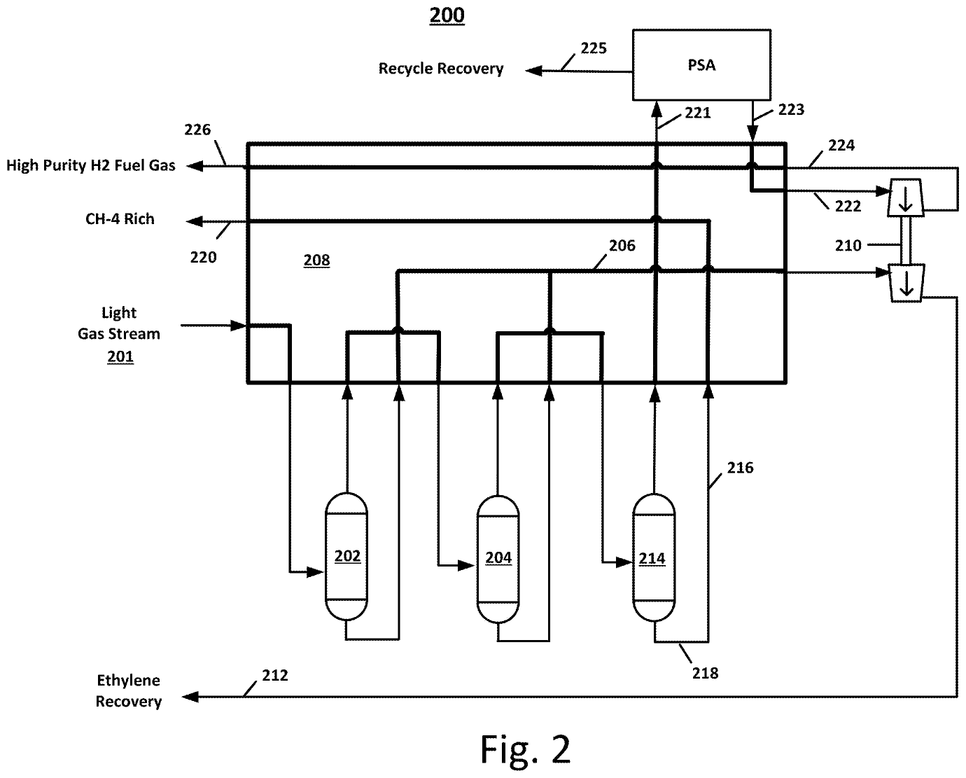

FIG. 2 is a schematic diagram illustrating some components of an example recovery section 200 for separating cracked gas 130. The cracked gas stream 130 from furnace system 100 may include H2, CH4, and ethylene. It should be noted the temperatures and pressures in this description are only illustrative of an example of the process. Depending on design constraints and considerations, the temperatures/pressures may vary, as will be appreciated by a person of skill in the art.

In examples, stream 130 may be further separated into light gas stream 201 that contains H2, CH4 and some of the ethylene, and streams containing the ethylene and heavier product streams. In examples, light gas stream 201 is progressively cooled, and separators (e.g., knock-out drums) 202 and 204 may be used to remove ethylene. The bottom streams of the knockout drums 202 and 204, which are enriched in ethylene, can be combined into an ethylene-rich stream 206. The ethylene-rich stream 206 can be recompressed using a turbo expander/compressor 210 to provide an ethylene-rich ethylene recovery stream 212.

In examples, the top streams of the knock-out drum 204, may be enriched in CH4 and H2 and can be further cooled in the cold box and provided to a third knock-out drum 214. The temperature of the third knock-out drum 214 may be about −163° C.±10° C. In examples, the top stream 216 from the third knock-out drum 214 may include an enriched H2 stream. The bottom stream 218 may include an enriched CH4 stream. In examples, the CH4-enriched bottom stream 218 may be reheated in the cold box and exits the system as a CH4-rich stream 220.

In examples, by controlling the temperature of the third knock-out drum 214 it may be possible to determine how much CH4 is knocked out. In examples, by determining how much CH4 is knocked out it may be possible to control the purity of the H2-enriched stream.

In examples, the H2-enriched top stream 216 may be reheated in the cold box to provide H2-enriched stream 221. Stream 221 may be separated into a high purity hydrogen stream 223 and reject or recycle recovery stream 225. Reject stream 225 may be recycled for recovery of contained hydrogen.

High purity hydrogen stream 223 may be cooled in cold box to provide a cooled high purity hydrogen stream 222. According to some examples, the temperature of the cooled high purity hydrogen stream 222 can be about −140° C.±10° C. and its pressure can be about 20 to about 35 barg. In examples, cooled high purity hydrogen stream 222 may be expanded using the turbo expander/compressor 210 to yield an expanded high purity hydrogen stream 224. This drops the temperature and pressure of the expanded high purity hydrogen stream 224. For example, according to some examples, the temperature of expanded high purity hydrogen stream 224 may be about −177° C.±10° C. and the pressure may be less than about 10 barg, for example about 6 barg. The expanded high purity hydrogen stream 224 may then be reintroduced to the cold box 208, thereby providing a cold stream within the cold box capable of providing an adequate temperature approach to effect the separation of CH4 and H2 in the knockout drum 214.

In examples, the expanded high purity hydrogen stream 224 may ultimately exit the cold box as recovered high purity hydrogen stream 226 and can be sent back to furnace system 100 as fuel for the burners. In examples, the recover section process 200 may recover over 95% or over 97% of the H2 available in the cracked gas stream 201. For purposes of this description, high purity hydrogen stream refers to a stream that includes greater than 95 mol % H2, or greater than 97 mol % H2, or greater than 98 mol % H2, with most of the remainder comprising CH4. In examples, the high purity hydrogen stream 226 may include a pure hydrogen stream with a purity >98 mol % hydrogen, for example about 98.5 mol % or more hydrogen, for example about 99 mol % or more hydrogen, for example about 99.5 mol % or more hydrogen.

In examples, the use of the recovered high purity hydrogen stream 226 as fuel for furnace system 100 may not provide the sufficient fired duty for the furnace system 100. For example, at 100% recovery, the recovered high purity hydrogen stream may not be sufficient to meet the required fired duty for furnace system 100 even when maximum combustion air preheating against flue gas is applied. Accordingly, in examples, the use of recovered high purity hydrogen stream, while beneficial, additional import of hydrogen to cover the fired duty shortfall may be required. Hydrogen importation can be expensive or unavailable in some locations. As such, it is advantageous to have a system and method that may be capable of balancing the fired duty to avoid importation of hydrogen.

Maximum combustion air preheat against flue gas may reduce required fired duty by about 25%, which may allow use of only internally produced H2-rich fuel gas (at about 95 mol % or lower) to be used to satisfy furnace duty demand. However, to eliminate CO2 emissions from the furnace may require pure hydrogen. Yet, internally generated high purity hydrogen can only cover about 85% of furnace duty (assuming 100% recovery and max air preheat). In examples, the process and system described herein address this shortfall by applying electric heating to the combustion air. In this manner, a further 15% reduction in duty may be possible to allow zero CO2 emission cracking while using only internally generated hydrogen.

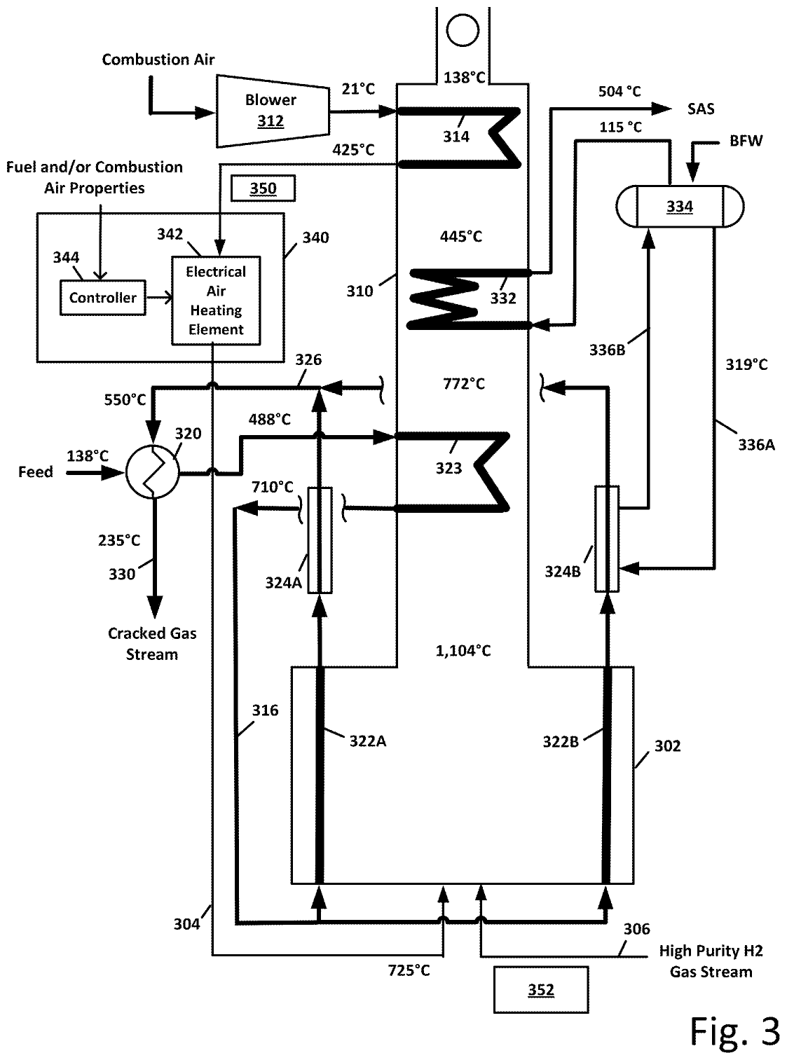

FIG. 3 is a schematic diagram illustrating some components of an example net zero furnace system 300 that is configured for net-zero steam-cracking. In examples, the furnace system 300 may be configured to use high purity hydrogen stream as the primary and/or sole fuel source in combination with combustion air. In examples, the high purity hydrogen stream may be a recovered high purity hydrogen stream from the furnace effluent. In examples, the high purity hydrogen stream may be provided from a source other than the cracked gases of the furnace system 300. In examples, the high purity hydrogen stream may be a recovered high purity hydrogen stream as described with reference to FIG. 2. In examples, the furnace system 300 may be configured to balance the fired duty of the system based on the high purity hydrogen stream.

In examples, furnace system 300 may include a radiant section 302 where a combustion mixture of preheated combustion air provided by line 304 and high purity hydrogen stream such as, for example, recovered high purity hydrogen stream 226 provided by recovery section 200 is burnt in burners (not shown). In examples, furnace system 300 may include burners that are suitable for high purity hydrogen. In examples, having a burner system suitable for high purity hydrogen may be needed to optimize the mixing of hydrogen and air to achieve complete combustion since hydrogen combustion can be sensitive to the combustion air/fuel ratio. In examples, hydrogen may have a high specific energy content per unit mass, so burners designed for hydrogen may need to manage the increased heat release compared to conventional fuels such as methane. In examples, hydrogen can cause embrittlement in some metals, so burner components may need to be made from metals that can be compatible with hydrogen to ensure safe and reliable operation.

In examples, furnace system 300 may include a convection section 310. In examples, heat from hot flue gas from radian section 302 can be recovered in the convection section 310 for one or more heating processes. In examples, the flue gas in convection section 310 may be used to preheat the feed in a feed preheater 323. In examples, heat recovered from convection section 310 may be used to create steam. In examples, the flue gas may be used to heat steam in a steam superheater 332 to produce super-heated steam (SHS). In the illustrated example, the flue gas in convection section 310 of furnace system 300 may be used to preheat the combustion air in an air preheater 314. In examples, additional and/or other heating may be provided via convection section 310.

In examples radiant section 302 may operate at about 1104° C., and the temperatures in the convection section 310 drop as the flue gas rises. It should be appreciated that the illustrated temperatures are only illustrative and other examples may use other temperatures.

In examples, a feed, comprising steam and an ethane-rich hydrocarbon may be provided to the radiant section 302 of the furnace system 300 for the olefin production. In examples, the feed may enter radiant section 302 via feed a line 316. In examples, the system and method herein provide for preheating the feed prior to injection into the radiant section of furnace system 300. In examples, the feed may be first preheated using a feed/effluent exchanger 320. In examples, the feed/effluent exchanger 320 may be configured to transfer heat from an effluent of the radiant section 302 and the feed. In examples, the effluent of the radiant section 302 may undergo one or more cooling processes prior to exchange heat with the feed via feed/effluent exchanger 320.

In examples, the feed may secondarily be heated via convection section 310 of furnace system 300. For example, the feed may be made to flow through a pipe preheater 323 that traverses convection section 310 to thus heat the feed. In examples, the combined feed preheating may be configured to preheat the feed to a temperature to 650-750° C. In the illustrated example, the feed may be preheated to a temperature of about 710° C. In examples, the preheated feed may be then passed through radiant section 302 of furnace system 300 via one or more tube banks 322A and 322B. In examples, the cracking reaction may occur in the one or more tube banks 322A and 322B. Although, two tube banks 322A and 322B are illustrated in the drawing, it should be appreciated that more or fewer tube banks may be used.

In examples, the cracked gas streams may exit tube banks 322A and 322B. In examples, the cracked gas streams may be cooled in one or more primary quench exchangers 324A and 324B, respectively. The cracked gas streams may be then combined to form gas stream 326, which may be used to preheat the incoming feed stream in the feed/effluent exchanger 320. In examples, the system and method herein may be configured to control the cooling of the cracked gas streams in primary quench exchangers 324A and 324B. In examples, to help balance the fired duty of the furnace system 300, the system and method may control the cooling of the cracked gas streams to increase the heat that may be transferred to the feed stream. In examples, the system and method as described may be configured to adjust the heat exchange in primary quench exchangers 324A and/or 324B to ensure that sufficient heat is transferred to feed to reach a superheated state.

In examples, after passing through feed/effluent exchanger 320 the cracked gas stream 330 may be separated into a light gas 130 and heavier gas. The light gas 130 may be processed in recovery section 200 as more fully described above to yield recovered high purity hydrogen stream 226, which in turn is mixed with preheated combustion air and combusted in radiant section 302.

In examples, saturated steam may be provided to the steam superheater 332 by heating BFW from steam drum 334. In examples, the quench exchangers 324A and 324B can be used to heat the BFW to provide the steam, which may be subsequently superheated to provide the SHS. In the illustrated example, BFW may be provided to the quench exchangers 324A and 324B via line 336A and the partially vaporized BFW/steam may be returned to the steam drum 334A via line 336B. Similar piping to and from the quench exchanger 324A is omitted for clarity.

In examples, combustion air may be provided to radiant section 302. In examples, to at least partially balance the fired duty of the system when using a high purity hydrogen stream as fuel, the furnace system 300 may preheat the combustion air prior to feeding it to the radiant section 302. In examples, the preheating of the combustion air may include the use of one or more electric heaters controlled based on the combustion air properties, the high purity hydrogen stream properties, or both.

In examples, combustion air may be provided via a line 304. In the illustrated furnace system 300, a blower 312 may be employed to provide combustion air at a temperature of about 21° C. (10-40° C.). In examples, the combustion air may be free or substantially free (i.e. no more than trace amounts) of hydrocarbons. According to some examples, the combustion air may be preheated in a first stage and in a second stage. In examples, combustion air may be preheated in a first stage via convection section 310. In examples, first heating may be conducted via air preheater 314 using heat from the flue gas in the convection section 310. In examples, air preheater 314 may include a conduit passing through convection section 310 through which combustion air may flow and thus heated. In examples, the first stage heating may preheat the combustion air to a temperature of about 425° C. (350-450° C.).

In examples, furnace system 300 may include a heater 340 for preheating combustion air prior to combustion. In examples, heater 340 is external to the furnace and/or to the convection section 310 and radiant section 302 of furnace system 300. In examples, heater 340 is an electric heater. In examples, the combustion air preheating via electric heater 340 may be provided as a second preheating stage. In examples, the combustion air preheated in a first stage may be fed to electric heater 340 to further preheat. In examples, combustion air preheated via preheater 314 in convection section 310 of furnace system 300 may be fed to the electric heater 340 for additional preheating prior to being injected into radiant section 310.

In examples, any power source may be used to power heater 340. In examples, heater 340 may be powered with green electricity. In examples, heater 340 may be powered using electricity provided by a renewable source such as solar, thermal, wind, or any combination thereof. In examples, the extent of combustion air preheat by heater 340 may be selected to balance with the available fired duty that recovered high purity hydrogen stream 226 can supply, to the point where no external hydrogen import is required. Accordingly, unlike furnace system 100, furnace system 300 may avoid importing supplemental hydrogen.

In examples, electrical heater 340 may include an electrical air heating element 342 and controller 344. In examples, controller 344 may include a microprocessor or microcontroller configured to execute instructions (e.g. executable instructions) stored in memory (not shown). In examples, electric air heating element 342 may include one or more resistive elements connected to a high voltage power source. Electrical current passes through the resistive elements and generates heat. In examples, the heat generated in the electric air heating element 342 may be transferred to the combustion air flowing over the resistive elements. The flow and/or amount of electrical current, and thus the heat generated in the resistive elements, can be adjusted by controller 344 based upon input provided to controller 344. By controlling the heat conveyed to the combustion air flowing over the resistive elements, controller 344 can control the temperature of the preheated combustion air that is subsequently mixed with high purity hydrogen stream 226 for combustion in the radiant section 302 of furnace system 300.

In examples, as shown in FIG. 3, an electric heater 340 may be controlled to pre-heat combustion air to about 725° C. (650-750° C.). However, other temperatures may also be used. In examples, controller 344 may be configured to control the temperature and adjust it up or down depending on one or more properties of the recovered high purity hydrogen stream 226. In examples, controller 344 may determine a preheating temperature for the combustion air based on the one or more properties of the high purity hydrogen fed to the radiant section 302 as fuel. In examples, controller 344 may then control heater 340 to preheat the combustion air to the preheating temperature it determined. In examples, controller 344 may be configured to control the temperature and adjust it up or down depending on the properties of the pre-preheated combustion air. In examples, controller 344 may adjust the temperature to accommodate changes in one or more properties of the recovered high purity hydrogen stream 226 and/or changes in one or more properties in the combustion air input to electric heater 340.

In examples, one or more sensors 350 may be configured to monitor properties of the combustion air input to electric heater 340 and/or combustion air output from electric heater 340. For example, one or more sensors 350 can measure temperature, flow rate, and/or pressure of combustion air entering electric heater 340 and/or exiting electric heater 340. In examples, controller 344 can use the one or more measured property values to adjust the electrical current provided to one or more resistive elements of electric air heating element 342. In examples, by adjusting the current provided to the resistive elements of electric air heating element 342, controller 344 may control the temperature of the combustion air exiting electric heater 340.

In examples, controller 344 may receive one or more properties related to high purity hydrogen stream 226, such as temperature, flow rate, pressure, hydrogen composition or fuel gas heating value. In examples, furnace system 300 may include one or more sensors 352 configured to measure one or more of properties related to the recovered high purity hydrogen stream 226. In examples, measured properties related to the recovered high purity hydrogen stream 226 may include, but not limited to, temperature, flow rate, pressure, percent composition, etc., of pure hydrogen stream 226. In examples, controller 344 may receive the measured property values or readings from the one or more sensors 352. In examples, sensors 350 and/or 352 may communicate the measured property values to controller 344 via wired connection and/or wirelessly. In examples, controller 344 may be configured to process the one or more measured properties and/or data received from sensors 350 and 352 to determine the required heat of the combustion air going into the radiant section 302 to ensure the duty is sufficient for the steam cracking. In examples, based on the determination of duty required, controller 344 may then trigger an adjustment in magnitude and/or flow of the electrical current provided to the one or more resistive elements of electric air heating element 342 to control the preheating of the combustion air.

In examples, electric heater 340 may be configured to increase the temperature of combustion air to compensate for a reduced fired duty provided by high purity hydrogen stream 226. In this manner the steam cracking may be carried out without having to import additional hydrogen for combustion.

In examples, the controller 344 may also control and/or be in communication with one or more second controllers that controls the feed pre-heating and/or the operation of the primary quench exchangers 324A and 324B, and the SHS production. In examples, the combination and coordination of these controls in furnace system 300 can provide additional compensation for a reduced fired duty.

### Experimental Data

The table below compares test results obtained for a conventional (Base) furnace design and fuel gas, a furnace design with hydrogen fuel gas and air preheat (H2-Rich with APH) like that shown in FIG. 1, a pure hydrogen fuel case using the same configuration as FIG. 1 with additional hydrogen import (Pure H2 with import, APH) and an example furnace design with a configuration based on one or more aspects of a furnace in furnace system 300 of FIG. 3 (Pure H2 no import +E-APH).

| | | | Pure H2 | Pure |
|---|---|---|---|---|
| | | | with | H2 no |
| | | H2-Rich | Import, | import + |
| Preliminary Balances | Base | with APH | APH | E-APH |
| Furnace Fired Duty, | 737.0 | 551.7 | 547.5 | 468.9 |
| Gcal/h | | | | |
| SHP Steam generation, | 556914 | 301124 | 295712 | 258801 |
| kg/h | | | | |
| Combustion air, kg/h | 1050420 | 745357 | 718313 | 615130 |
| Electric Air Preheat | | | | 57.6 |
| (425 C. -> 725 C.), MWh | | | | |
| Fuel Gas to furnaces, | 39318 | 23372 | 19111 | 16367 |
| kg/h | (85 mol | (95 mol | (100% | (100% |
| | % H2) | % H2) | H2) | H2) |
| PSA H2 recovered from | | | 16553 | 16367 |
| cracker effluent, kg/h | | | | |
| H2 import required | | zero | 2558 | zero |
| Direct CO2 | 63666 | 19098 | 225 | |
| Emission—Furnaces | | | | |
| & H2 unit, kg/h | | | | |
| Total Direct | 0.297 | 0.089 | 0.001 | 0.000 |
| CO2 emission, | | | | |
| kg CO2/kg Ethylene | | | | |
| Steam Import | 3977 | (40457) | (44357) | 1866 |
| (Export)—Cracker + | | | | |
| H2 unit, kg/h | | | | |
| Power import—Cracker + | 8886 | 71331 | 72871 | 128899 |
| H2 unit, kWh | | | | |

While this disclosure is described in the context of an ethane cracker, application of electric air preheat to reduce or eliminate external hydrogen import is not limited to ethane cracking furnaces only, but can also be applied to furnaces that process other gas or liquid feedstocks. When ranges are disclosed herein, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

It is understood that the present subject matter may be embodied in many different forms and should not be construed as being limited to the examples set forth herein. Rather, these examples are provided so that this subject matter will be thorough and complete and will convey the disclosure to those skilled in the art. Indeed, the subject matter is intended to cover alternatives, modifications, and equivalents of these examples. Furthermore, in the detailed description of the present subject matter, numerous specific details are set forth in order to provide a thorough understanding of the present subject matter. However, it will be clear to those of ordinary skill in the art that the present subject matter may be practiced without such specific details.

Aspects of the present disclosure are described herein with reference to flowchart illustrations and/or block diagrams of methods, apparatuses (systems), and computer program products according to examples of the disclosure. It will be understood that each block of the flowchart illustrations or block diagrams, and combinations of blocks in the flowchart illustrations or block diagrams, may be implemented by one or more apparatuses that create a mechanism for implementing the functions/acts specified in the block or blocks of the flowchart or block diagram.

The above description sets forth numerous specific details such as examples of specific systems, components, methods, and so forth, in order to provide a good understanding of several examples of the present disclosure. It is to be understood that the above description is intended to be illustrative and not restrictive. Many other examples will be apparent to those of skill in the art upon reading and understanding the above description. The scope of the disclosure should, therefore, be determined with reference to the appended claims, along with the full scope of equivalents to which such claims are entitled.

What is claimed is:

1. A method comprising:
cracking a feed comprising hydrocarbon feedstock and steam in a furnace to create a cracked gas comprising ethylene and hydrogen;
preheating a stream of combustion air to yield a preheated combustion air stream;
mixing a high purity hydrogen stream with the preheated combustion air stream to create a combustion mixture; and
burning the combustion mixture in the furnace;
wherein the stream of combustion air is preheated to a temperature based on a property of the high purity hydrogen stream.

2. The method of claim 1 wherein the combustion air stream is heated by a heater that is external to the furnace.

3. The method of claim 2 wherein heating the combustion air comprises:
a resistive element transmitting an electrical current to generate heat for heating the stream of air;
receiving and processing data relating to the property of high purity hydrogen stream; and
adjusting the electrical current in response to receiving and processing the data relating to the high purity hydrogen stream property.

4. The method of claim 1 wherein the property relates to a percentage of hydrogen in the high purity hydrogen stream.

5. The method of claim 1 wherein the property relates to a flow rate of the high purity hydrogen stream.

6. The method of claim 1 wherein the property relates to a temperature of the high purity hydrogen stream.

7. The method of claim 1 wherein the property relates to a pressure of the high purity hydrogen stream.

8. The method of claim 1 wherein the stream of air is heated to the temperature that is based on two or more properties of the high purity hydrogen stream.

9. The method of claim 1 wherein the stream of air is heated to the temperature based on a property of the stream of air before it is heated.

10. The method of claim 1, further comprising separating the high purity hydrogen stream from the cracked gas.

11. An apparatus comprising:
a cracking furnace comprising tubes in which a feed comprising hydrocarbon feedstock and steam is cracked to create a cracked gas comprising ethylene and hydrogen; and
a heater external to the cracking furnace for preheating a stream of combustion air to a temperature based on a property of a high purity hydrogen stream to yield a preheated combustion air stream;
wherein the furnace is configured to mix and burn the high purity hydrogen stream and the preheated combustion air stream to heat the tubes.

12. The apparatus of claim 11 wherein the heater comprises:
an electric heating element for preheating the combustion air stream; and
a control system for adjusting a heat output of the electric heating element for preheating the combustion air stream;
wherein the control system is configured to receive and process data relating to the property of the high purity hydrogen stream, and
wherein the control system is configured to adjust the heat output of the electric heating element in response to receiving and processing the data relating to the high purity hydrogen stream property.

13. The apparatus of claim 12 wherein the electric heating element comprises a resistive element that generates heat when the resistive element conducts an electric current.

14. The apparatus of claim 13 wherein the control system comprises a microcontroller for processing the data based on executable instructions stored in memory.

15. The apparatus of claim 14 wherein the microcontroller is configured to adjust a flow of the electric current through the resistive element based on the processing of the data.

16. The apparatus of claim 11 wherein the property relates to a percentage of hydrogen in the high purity hydrogen stream.

17. The apparatus of claim 11 wherein the property relates to a flow rate of the high purity hydrogen stream.

18. The apparatus of claim 11 wherein the property relates to a temperature of the high purity hydrogen stream.

19. The apparatus of claim 11 wherein the property relates to a pressure of the high purity hydrogen stream.

20. The apparatus of claim 11 wherein the heater is configured to heat the stream of air to the temperature that is based on two or more properties of the high purity hydrogen stream.

21. The apparatus of claim 11 wherein the heater is configured to heat the stream of air to the temperature that is based on a property of the combustion air stream before it is preheated.

22. The apparatus of claim 11, further comprising a separator for separating the high purity hydrogen stream from the cracked gas.

23. A system for controlling a steam cracking furnace comprising:
a controller configured to control an electric heater to pre-heat a combustion air feed to a radiation section of the cracking furnace; and
one or more sensors configured to sense properties of a recovered pure hydrogen stream fed to the radiation section of the cracking furnace as fuel,
wherein the controller is configured to receive the sensed properties from the one or more sensors and control the electric heater based on the one or more properties sensed.

24. A method of controlling a steam cracking furnace comprising:
sensing one or more properties of a high purity hydrogen stream fed to a radiant section of a cracking furnace as fuel;
determining based on the sensed properties a preheating temperature for a combustion air feed to the radiant section of the cracking furnace; and
controlling an electric heater to preheat the combustion air feed to the preheating temperature prior to feeding the combustion air feed to a radiant section of the cracking furnace.
